(19) United States
(12) Patent Application Publication
(10) Pub. No.: US 2014/0073995 A1
(43) Pub. Date: Mar. 13, 2014

(54) HISTOTRIPSY THERAPY SYSTEM

(71) Applicants: Dejan Teofilovic, Ann Arbor, MI (US); Timothy L. Hall, Ann Arbor, MI (US); Charles A. Cain, Ann Arbor, MI (US)

(72) Inventors: Dejan Teofilovic, Ann Arbor, MI (US); Timothy L. Hall, Ann Arbor, MI (US); Charles A. Cain, Ann Arbor, MI (US)

(21) Appl. No.: 14/024,394

(22) Filed: Sep. 11, 2013

Related U.S. Application Data
(60) Provisional application No. 61/699,779, filed on Sep. 11, 2012.

(51) Int. Cl. A61B 17/22

(52) U.S. Cl. CPC A61B 17/22012
USPC 601/2

(57) ABSTRACT

A Histotripsy therapy system is provided that can include any number of features. In some embodiments, the system includes at least one signal switching amplifier electrically coupled to a high voltage power supply, a pulse generator electrically coupled to signal switching amplifier(s), at least one matching network electrically coupled to the signal switching amplifier(s), and an ultrasound transducer having at least one transducer element, each transducer element of the ultrasound transducer being coupled to the at least one matching network. In some embodiments, each transducer element has an input impedance that is higher, sometimes more than 2 times higher, than an output impedance of its corresponding signal switching amplifier. Methods of use are also described.

### CROSS REFERENCE TO RELATED APPLICATIONS

This application claims the benefit under 35 U.S.C. 119 of U.S. Provisional Patent Application No. 61/699,779, filed Sep. 11, 2012, titled “Histotripsy Therapy System”. This application is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure relates generally to systems for powering and controlling a therapy transducer. More specifically, this disclosure describes powering transducers at high voltages to perform Histotripsy therapy.

### BACKGROUND

Histotripsy applies high intensity focused acoustic energy at a very low duty cycle to homogenize cellular tissues. Histotripsy high intensity focused ultrasound is pulsed to induce acoustic cavitation in the target tissue. Acoustic cavitation occurs when rapid cycling from compression to rarefaction forms micro-bubbles which oscillate and collapse violently releasing tremendous energy as stresses and pressures. The resulting “bubble cloud” completely homogenizes the tissues.

In order to generate an effective bubble cloud through layers of tissue from an external focused ultrasound transducer, pressures generated by the transducer must be very high. High pressures are necessary to overcome tissue attenuation and non-linearity. Compared to water, the average tissue attenuates the sound wave 0.8 db/cm at 1 MHz. Different attenuation in various tissues causes non-linearity. In order to achieve the necessary pressure wave, the voltage applied to the transducer must exceed 0.6 kV and in some applications be as high as 1.8 kV. High ultrasound frequency (500 KHz-1.5 MHz) drive systems amplifiers utilize commercially available P-Mosfet transistors that are limited to 500V. These amplifiers generate a square wave. The peak to peak voltage on the square wave CANNOT exceed 500V and is typically limited to 400V to provide a safety margin. The amplifier output is matched to the ultrasound transducer elements through an LC (inductor-capacitor) matching network which also converts the square wave to a sinusoidal wave. The output impedance of these amplifiers is typically 20Ω and it is matched to transducer input through the LC matching network with the same input impedance of 20Ω.

Initial attempts to homogenize tissue through a significant tissue depth using a commercially available RF power amplifier were not optimal because the drive system voltage to the transducer was limited. Commercially available RF power amplifiers of reasonable size and cost do not produce a high enough peak power output for histotripsy. HIFU does not require high peak power because the therapeutic effect is produced by much longer acoustic bursts. Since Histotripsy requires such high pressure amplitudes and high peak power high voltage driven transducers, development of practical and cost effective Histotripsy Therapy Systems is challenging.

Medical devices are required to pass the international standard IEC60601 electrical safety test. It is well known in the field of therapeutic ultrasound systems that electromagnetic compatibility (EMC) is a challenge if not impossible to achieve for these devices. Lithotripsy devices are exempt from the EMC testing requirement because many thought that it was impossible for them to pass.

An important characteristic of Histotripsy is the absence of thermal injury at the target site and the absence of damage; thermal or mechanical, in the tissues between the skin surface and target tissue. Avoidance of thermal injury is a challenge in Histotripsy applications where the target tissue is deep and the acoustic path is obstructed by bones or other intervening objects that block acoustic energy transmission.

### SUMMARY OF THE DISCLOSURE

A Histotripsy therapy system is provided, comprising at least one signal switching amplifier electrically coupled to a high voltage power supply, a pulse generator electrically coupled to the at least one signal switching amplifier, at least one matching network electrically coupled to the at least one signal switching amplifier, and an ultrasound transducer having at least one transducer element, each transducer element of the ultrasound transducer being coupled to the at least one matching network, each transducer element having an input impedance that is more than 2 times higher than an output impedance of its corresponding signal switching amplifier.

In some embodiments, the system is configured to provide Histotripsy therapy comprising forming cavitational microbubbles within tissue at a focal zone of the transducer without damaging surface tissue. In some embodiments, the Histotripsy therapy can comprise generating a Histotripsy pulse having a pulse length less than 20 μsec, a peak negative pressure greater than 10 MPa, and a duty cycle less than 5%.

In one embodiment, the output impedance of the signal switching amplifiers is less than or equal to 50 ohms.

In another embodiment, the input impedance of each transducer element of the ultrasound transducer is between 10 and 500 ohms.

In some embodiments, the pulse generator provides a square wave voltage of up to 1000V. In one embodiment, the plurality of signal switching amplifiers convert the square wave voltage into a sinusoidal wave voltage that is 2 to 10 times greater than the square wave voltage. In some embodiments, the sinusoidal wave voltage is up to 5000 Vpp.

In one embodiment, the at least one transducer element is configured to be driven at up to 4 MHz.

In another embodiment, the at least one matching network is configured to match or exceed the output impedance of the at least one signal switching amplifier with a combined impedance of the at least one matching network and at least one transducer element.

In some embodiments, the ultrasound transducer has a diameter of ranging from 2 cm to 50 cm.

In some embodiments, the system further comprises an emission suppression circuit that includes inductors placed in series with gates of mosfet transistors disposed in the at least one signal switching amplifier, and further includes capacitors placed in parallel with an output of the at least one signal switching amplifier, the inductors and capacitors being configured to reduce electromagnetic emissions of the at least one signal switching amplifier. In one embodiment, the inductors and capacitors are configured to eliminate spikes at a beginning and end of a square wave pulse.

In some embodiments, the ultrasound transducer has an F-number between 0.4 and 1.2.

A Histotripsy therapy system is provided, comprising at least one signal switching amplifier electrically coupled to a high voltage power supply, a pulse generator electrically coupled to the at least one signal switching amplifier, at least one matching network electrically coupled to the at least one signal switching amplifier, and an ultrasound transducer having at least one transducer element, each transducer element of the ultrasound transducer being coupled to the at least one matching network, each transducer element having an input impedance that is higher than an output impedance of its corresponding signal switching amplifier so as to deliver a Histotripsy pulse having a pulse length less than 20 μsec, a peak negative pressure greater than 10 MPa, and a duty cycle less than 5% to tissue.

In some embodiments, the output impedance of the at least one switching amplifier is less than 50 ohms.

In other embodiments, the impedance of the at least one transducer element is greater than 50 ohms.

In some embodiments, the at least one matching network matches a real impedance of the at least one transducer element to the output impedance of the at least one signal switching amplifier and reduces an imaginary impedance of the at least one transducer element.

An ultrasound therapy system is provided, comprising a high voltage power supply, a pulse generator, a plurality of signal switching amplifiers electrically coupled to the high voltage power supply and the pulse generator, a plurality of matching networks, each matching network being electrically coupled to one of the plurality of signal switching amplifiers, and an ultrasound transducer having a plurality of transducer elements, each transducer element of the ultrasound transducer being electrically coupled to one of the plurality of matching networks and one of the plurality of signal switching amplifiers, wherein each transducer element has an input impedance that is higher than an output impedance of its corresponding signal switching amplifier.

In some embodiments, the input impedance of each transducer element is at least 2 times higher than the output impedance of each corresponding signal switching amplifier.

In one embodiment, the input impedance of each transducer element is at least 100Ω and the output impedance of each signal switching amplifier is less than or equal to 50Ω.

In some embodiments, the ultrasound transducer has an F-number less than 1.2.

In one embodiment, the system comprises up to 36 signal switching amplifiers, up to 36 matching networks, and up to 36 transducer elements.

In some embodiments, the plurality of matching networks are configured to bring the combined impedance of the plurality of matching networks and the plurality of transducer elements down to match that of the plurality of signal switching amplifiers.

In another embodiment, the plurality of signal switching amplifiers each include an emission suppression circuit configured to reduce spikes on a square wave pulse output of the signal switching amplifiers.

A method of delivering Histotripsy ultrasound therapy is also provided, comprising generating in a pulse generator a Histotripsy pulse having a pulse length less than 20 μsec, a peak negative pressure greater than 10 MPa, and a duty cycle less than 5%, amplifying the Histotripsy pulse from the pulse generator with a plurality of signal switching amplifiers, impedance matching an input impedance of a plurality of ultrasound transducers to an output impedance of the plurality of signal switching amplifiers with a plurality of matching networks, and delivering the Histotripsy pulse to tissue with the plurality of ultrasound transducers.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a block diagram of one embodiment of a Histotripsy therapy system.

FIG. 2 is a block diagram of components for another embodiment of a Histotripsy therapy system.

FIG. 3 illustrates one embodiment of a Histotripsy therapy transducer.

FIG. 4 illustrates a matching network according to one embodiment.

FIGS. 5A-5D illustrate various mathematical outputs of the Histotripsy therapy system.

FIG. 6 shows a matching network converting high voltage square wave pulse into sinusoidal waves.

FIGS. 7A-7B illustrate emissions coming from the Histotripsy therapy system.

FIG. 8 illustrates the peak of emissions identified at a specific frequency.

FIG. 9 illustrates an amplifier according to one embodiment.

FIGS. 10A-10B illustrate one embodiment of a mosfet amplifier eliminating spikes in the beginning and end of a square wave output.

FIGS. 11A-11B also show radiated emissions from a system.

FIG. 12 shows one embodiment of a transducer configured to treat thyroid or other tumors.

FIG. 13 illustrates another transducer configured to perform Histotripsy therapy.

### DETAILED DESCRIPTION

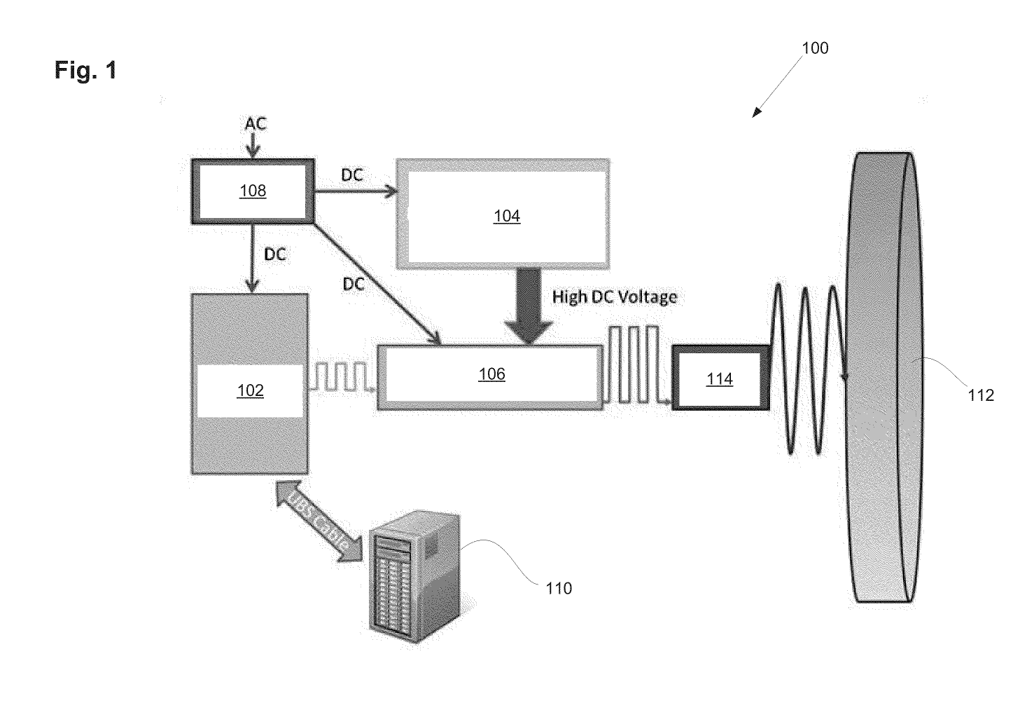

The primary components of one embodiment of a Histotripsy therapy system 100 are illustrated in the block diagram of FIG. 1. The system 100 can include a pulse generator 102, high voltage power supply 104, and signal switching amplifier 106 which are powered by AC to DC power supply 108. The pulse generator 102 can be controlled with or by a computer or controller 110 which is configured to set the ultrasound frequency, pulse duration and pulse repetition frequency of the system 100. The computer can also control the output of high voltage power supply 104. In some embodiments, the high voltage power supply can be a 500V power supply with an adjustable range of approximately 0-400V. Output from the pulse generator 102 can be amplified by the signal switching amplifier 106 up to the voltage of the power supply output. The output from signal switching amplifier can be impedance matched with the input impedance of transducer 112 through matching network 114. The matching network can be configured to bring the combined impedance of the matching network and the transducer down to match that of the amplifier(s).

The ultrasound therapy transducer 112 of FIG. 1 can be configured to generate Histotripsy pulses to deliver Histotripsy therapy to tissue. Histotripsy uses controlled cavitation bubble clouds to induce mechanical tissue fractionation. Histotripsy bubble clouds can be produced by delivering Histotripsy energy to tissue with a Histotripsy transducer, defined by using short (<20 μsec), high pressure (peak negative pressure>10 MPa) shockwave ultrasound pulses at a low duty cycle, typically <5%, minimizing thermal effects. Based on the high echogenicity of cavitating bubble clouds, treatment can also be readily monitored in real time using any conventional ultrasound imaging system, allowing the operator to acknowledge whether cavitation bubble clouds have been generated.

In order to generate a high voltage signal to drive the transducer, the impedance of each transducer element of ultrasound transducer 112 should be relatively high (e.g., at least 100-200Ω±25Ω) with respect to the impedance of its corresponding amplifier and matched with the matching network to an output impedance of the amplifier 106 that is less than 50Ω) and preferably less than 20Ω. In some embodiments, the output impedance of the amplifier can range from 10-50Ω. Lower than 10Ω would load the amplifier possibly causing overheating and damage, and higher than 50Ω would provide less amplifying capabilities. To put it more simply, each transducer element of the ultrasound transducer should have an input impedance that is higher, and preferably more than 2× higher than an output impedance of the corresponding signal switching amplifier that provides amplified signals to the transducer element.

The high input impedance of the transducer elements of this system, combined with low output impedance of the amplifiers, enables the oscillating matching network to convert a square wave voltage to a sinusoidal voltage and significantly amplify it. These high sinusoidal voltage inputs to the transducer elements are essential to generate the high pressures required for Histotripsy.

The relatively large difference in the output impedance of the amplifier 106 and the transducer impedance requires the use of a matching network 114. The matching network can include an inductor L and capacitor C (LC) circuit that oscillates the signal, and converts the square wave from the amplifier into a sinusoidal voltage that is significantly amplified. A transformer circuit can be employed to accomplish the same conversion from square wave to sinusoidal voltage. In this configuration, for example, a 350V power input to the signal switching amplifier outputs a 350V square wave that can be converted to a sinusoidal wave with amplitudes between 1,200 and 1,600 Vpp.

Transducers used for non-invasive Histotripsy of deep tissue targets require large apertures (diameter) and relatively long focal length. A typical transducer operating at 750 KHz may have a 13 cm aperture and 11 cm focal length (f=0.85). A single element transducer of this dimension would therefore have very low impedance. A multiple element transducer would also have very low impedance if all of the elements were driven by a single amplifier. When these low impedance transducer systems are driven by a single amplifier, the acoustic power necessary to perform Histotripsy is not achieved. Histotripsy therapy systems according to various embodiments herein can utilize multi-element transducers with each element having an impedance of approximately 100-200Ω±25Ω and each transducer being driven be a separate signal switching amplifier and matching network. These multi-element transducers driven by multiple switching amplifier and matching network generate the high power required for performing Histotripsy.

The nature of the pulses generated to drive Histotripsy therapy transducers emits radiation that must be filtered to pass the EMC component of the IEC60601-1-2 tests required for electrical medical devices in the United States and most other countries. Most of the interference is emitted from the multichannel amplifiers where the mosfet transistors are located. The electrical spikes that create the interference and high frequency oscillations that follow can be reduced by adding snubber capacitors at the output of the mosfet amplifiers and ferrite inductors very close to the gates of the mosfet transducers. Other more common essential components and methods are also employed such as metal enclosure and tuning the operating frequency to the anti-resonant frequency of the transducer.

Avoidance of unintended thermal injury at the focal point and in the tissues between the skin surface and focal point (pre-focal) is achieved by designing Histotripsy transducers to have the lowest possible F-Number, which is defined as the focal length divided by the aperture (transducer diameter). The F-number of Histotripsy transducers must be less than 1.2.

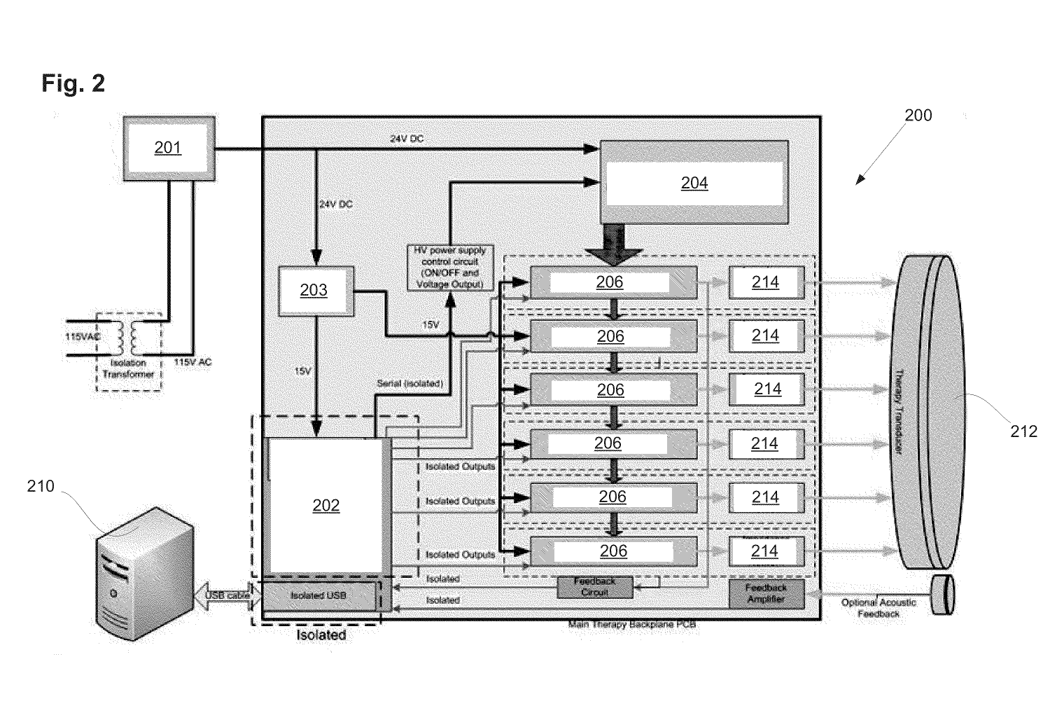

In the embodiment of FIG. 2, a Histotripsy therapy system 200 can be connected to a medical grade power supply 201 and can include a multiple-element transducer 212 having a plurality of amplifiers 206 and matching networks 214 corresponding to each transducer element. For example, in one embodiment, the system can include a 36 element transducer coupled to 36 amplifiers and 36 matching networks. Alternatively, the system could include 6 amplifiers each having 6 channels, with each channel connected to a matching network. In one embodiment, the transducer can be driven at an ultrasonic frequency of up to 750 KHz.

In one embodiment, the medical grade power supply 201 can provide 24V of DC class 2 medical grade power to the system. The system can include a DC/DC 24V to 15V converter 203, which provides the power to a pulse generator 202 that includes an isolated DC/DC 3.3V converter. The single pulse generator 202 can be configured to send signals to all of the amplifiers 206, which are powered by a single high voltage power supply 204. The system can be controlled by a computer or controller 210, which can be connected electronically to the system (e.g., by USB).

In the embodiment of FIG. 2, each transducer element of the ultrasound transducer 212 is coupled to its own corresponding matching network 214, which is coupled to its own amplifier 206 (or amplifier channel). As described above, the system is optimized when each transducer element has an impedance that is more than 2 times higher than an output impedance of its corresponding signal switching amplifier (or amplifier channel).

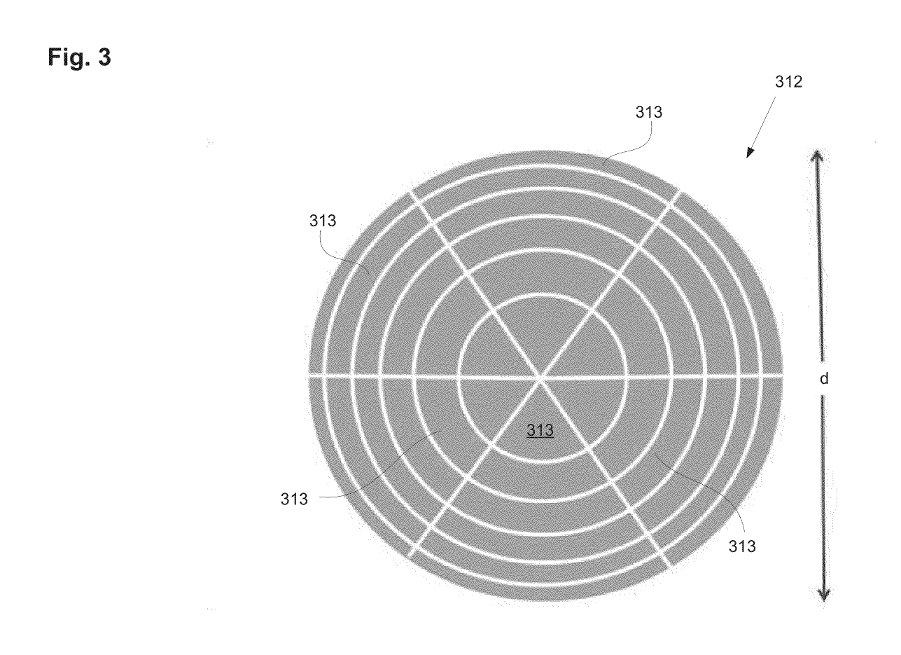

FIG. 3 illustrates one embodiment of a Histotripsy therapy transducer 312. In this illustrated embodiment, the transducer can include a plurality of transducer elements 313 (not all elements 313 are labeled in FIG. 3 for ease of illustration). In one specific embodiment, there can be 36 transducer elements 313, each of the 36 transducer elements embodiment having an average working impedance in the range of 130-180Ω. Each element can also have approximately the same surface area, as shown. This particular embodiment comprises a circular array with a diameter d. In some embodiments, the diameter d can be approximately 12.5 cm (aperture) and have a 11 cm focal length, resulting in an F-number of 0.88. The resulting active surface area is 120.2 cm² (excluding 0.5 mm gap between elements) and each element is approximately 3.34 cm². Such multi-element transducers used for Histotripsy can be targeted to a fixed focus, or they can be used in a phased array and the focus moved with electronic steering. The focus point of fixed focal length transducers can be changed by mechanically positioning the transducer using a micromanipulator or other methods.

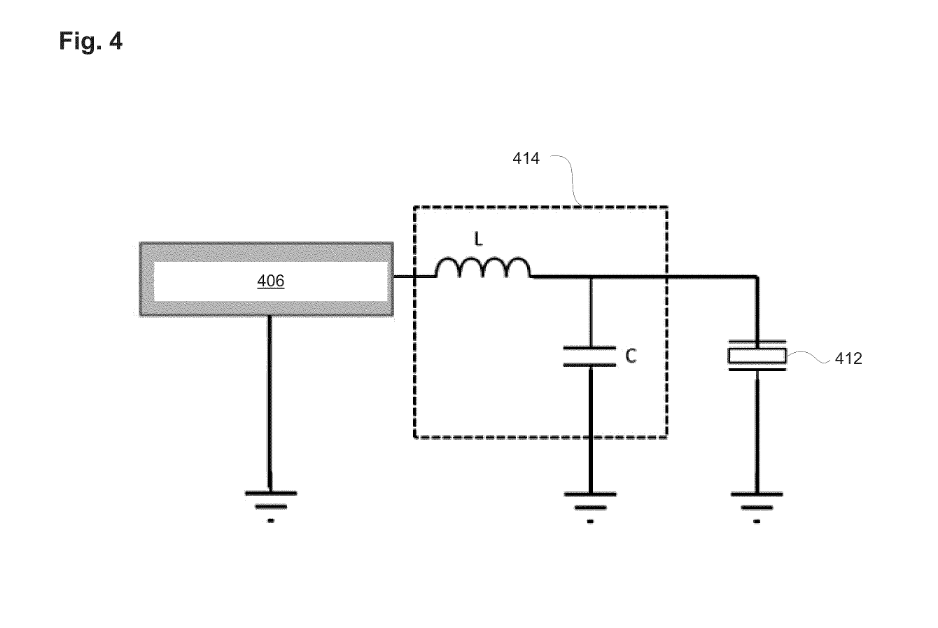

FIG. 4 illustrates one embodiment of a matching network 414, which can be the matching networks shown in FIG. 2 above. The matching network can be an LC circuit configured to bring the combined impedance of the matching network and the Histotripsy transducer 412 down to match that of the amplifier(s) 406 of the Histotripsy therapy system.

In one embodiment of a Histotripsy therapy system, the output impedance of the amplifiers (such as amplifiers 206 in FIG. 2) is approximately 14Ω. The impedance matching network 414 (or network 214 in FIG. 2) can be an LC circuit. The inductor L and capacitor C are selected to bring the combined impedance of the matching network and transducer element down to 14Ω to match that of the amplifier output impedance. To optimally match impedance in this example, the inductance L=18 micro-Henry (μH) and the capacitance C=2.2 nano Farad (nF). These values are calculated using matlab and the following formulas using a custom “Quick Matching Network Calculator”.

The average transducer element impedance in this example can be: 157-233j. The working frequency of the transducer can be 0.7 MHz. The matlab inputs and are shown in Equation 1, which follows:

%Inputs

Ze=157-233j;

f0=0.7e6;

%Capacitance range

C=[100e-12:100e-12:10e-9];

w0=2*pi*f0;

Zc=1./(j*w0*C);

Zp=Zc*Ze./(Zc+Ze);

Matlab Figure(1);

plot(C*1e9,real(Zp));

hold on;

plot(C*1e9,imag(Zp),‘r’);

xlabel(‘Capacitance(nF)’);

ylabel(‘Impedance’);

legend(‘Real’,‘Imaginary’);

Matlab Figure(2);

L=−j*imag(Zp)/(j*w0);

plot(C*1e9,L*1e6);

xlabel(‘Capacitance(nF)’);

ylabel(‘Inductance(\muH)’);

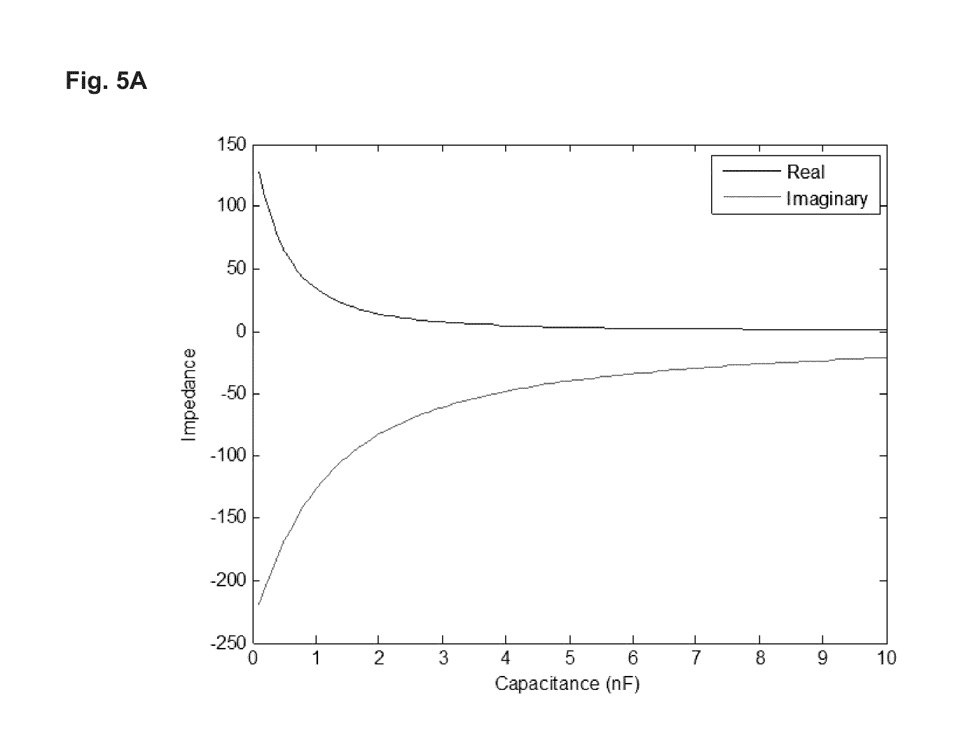

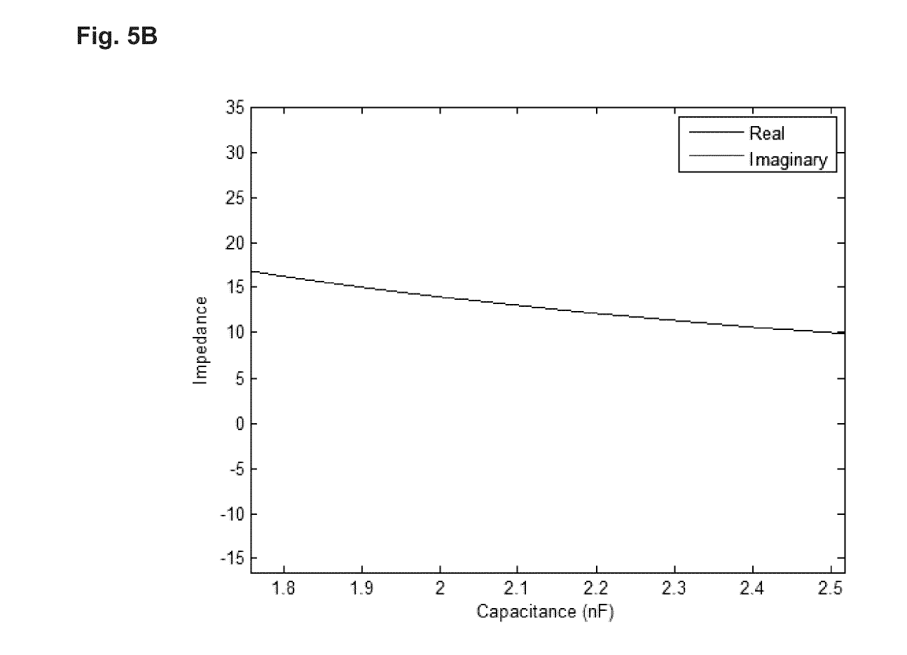

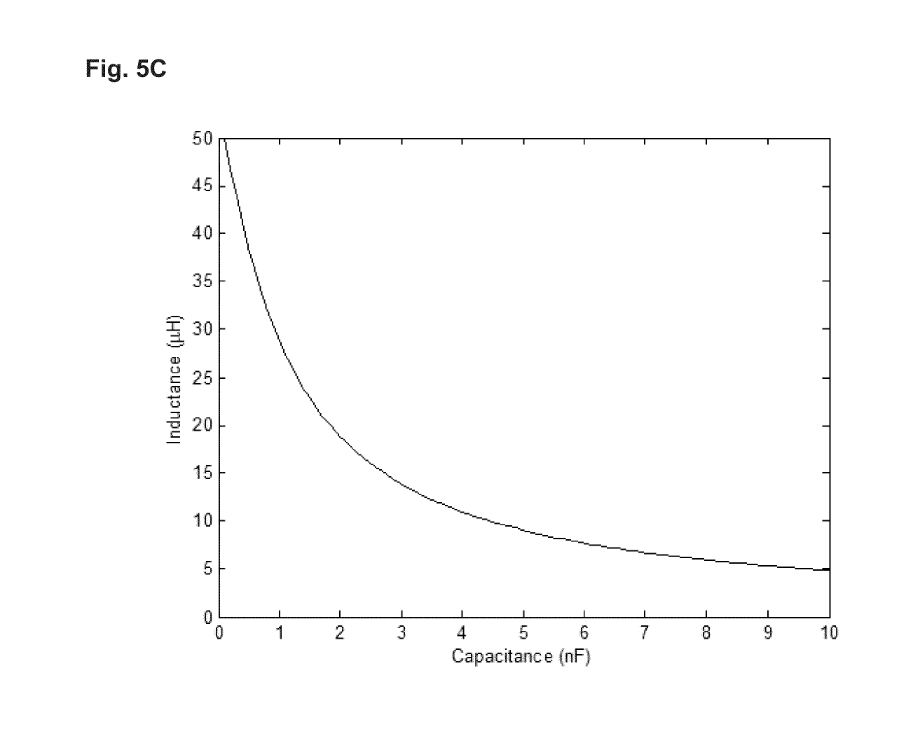

The Matlab outputs can be illustrated as graphs in FIGS. 5A-5D, which are used to select capacitance and inductance values for the matching network. The first graph, shown in FIG. 5A, plots impedance against capacitance. The second graph, FIG. 5B, is FIG. 5A zoomed to show the impedance range between 10 and 17Ω. A 2.2 nF capacitor can be used to lower the impedance of the transducer element and matching network to 14Ω.

The third graph, FIG. 5C, plots impedance against capacitance and is used to determine the inductor value which cancels imaginary impedance to make it as close to zero as possible. The fourth graph, FIG. 5D, zooms FIG. 5B to show the inductance range between 13 μH and 25 μH. In this embodiment, the optimal inductance match with 2.2 nF capacitance is 18 μH.

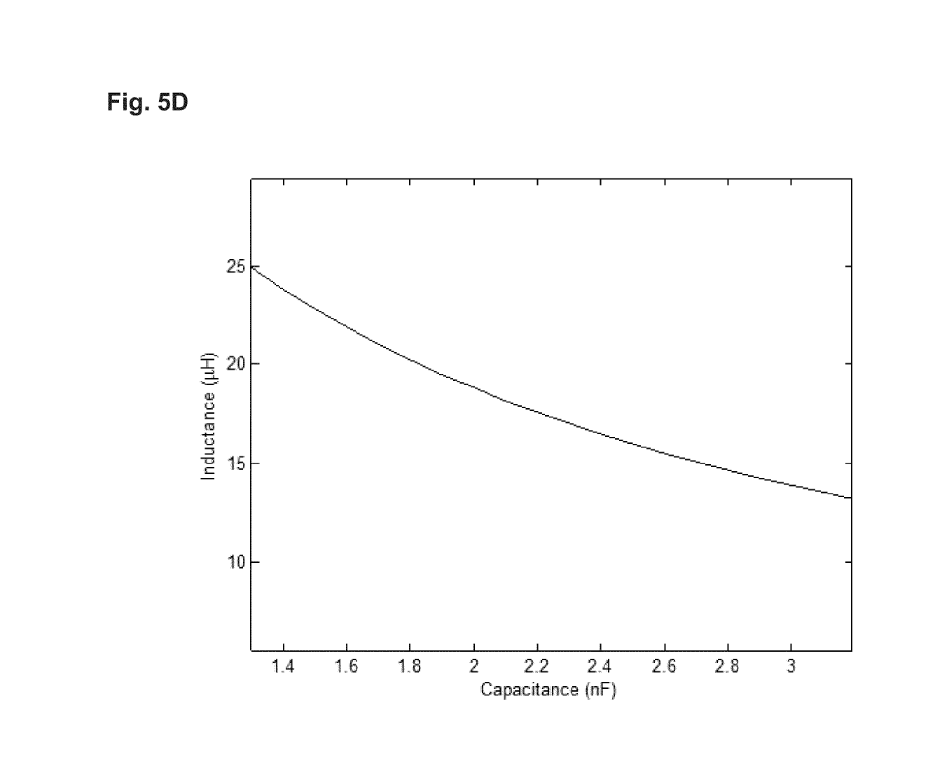

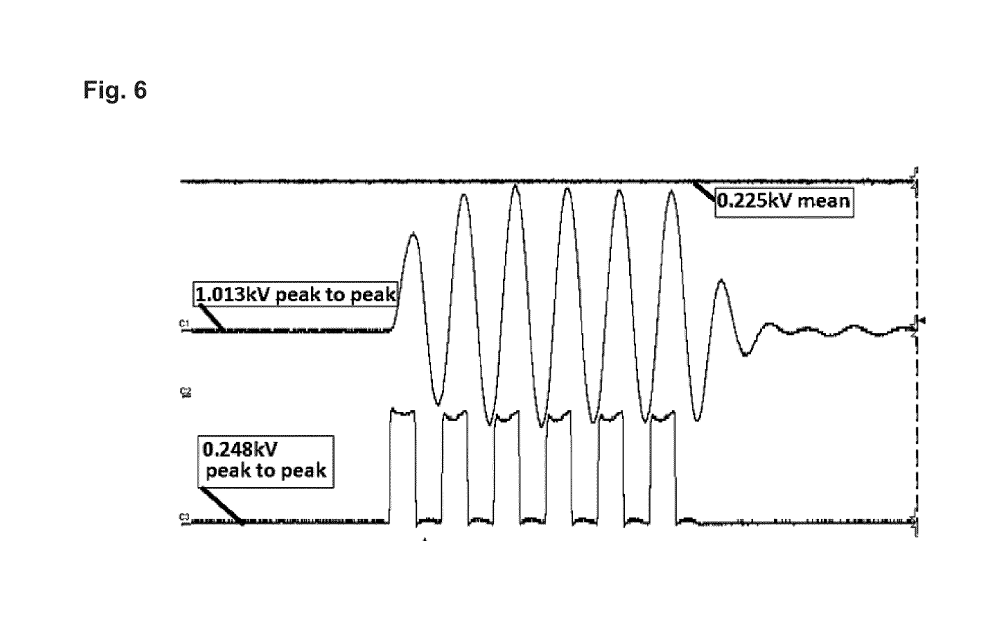

The matching network described above converts a high voltage square wave pulse up to 350V peak to peak (voltage applied from the power supply) to a sinusoidal wave that is amplified 4.5× up to 1,600V. In the example illustrated in FIG. 6, the signal switching amplifiers output a square wave of 0.248 kV and the voltage measured across the transducer element is 1.013 kVpp. This is an L-type high pass impedance matching network is a resonant circuit for a systems in which the load impedance much higher than the input impedance of the transducer element.

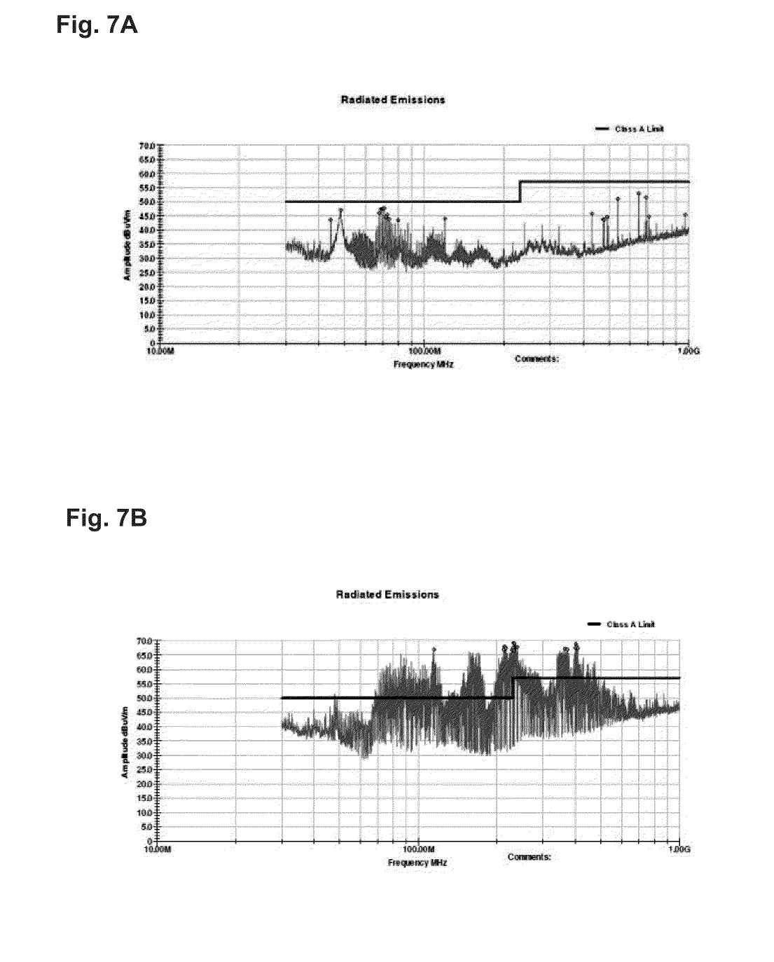

Significant electromagnetic emissions were encountered when the Histotripsy therapy system of FIG. 2 was first evaluated for IEC60601-1-2 Electromagnetic Compatibility at a certified testing lab. Two operational modes were tested: Standby; where the entire system is turned on including the imaging system and is ready for Histotripsy therapy pulsing, shown in FIG. 7A, and Normal Mode; where the system is turned on as above and the Histotripsy therapy pulsing is turned on to maximum power, shown in FIG. 7B. The system radiated emission in the Normal Mode only; hence it was determined the emissions come from the Histotripsy therapy system. It can be seen in FIG. 7B that the radiated emissions exceed the Class A limit.

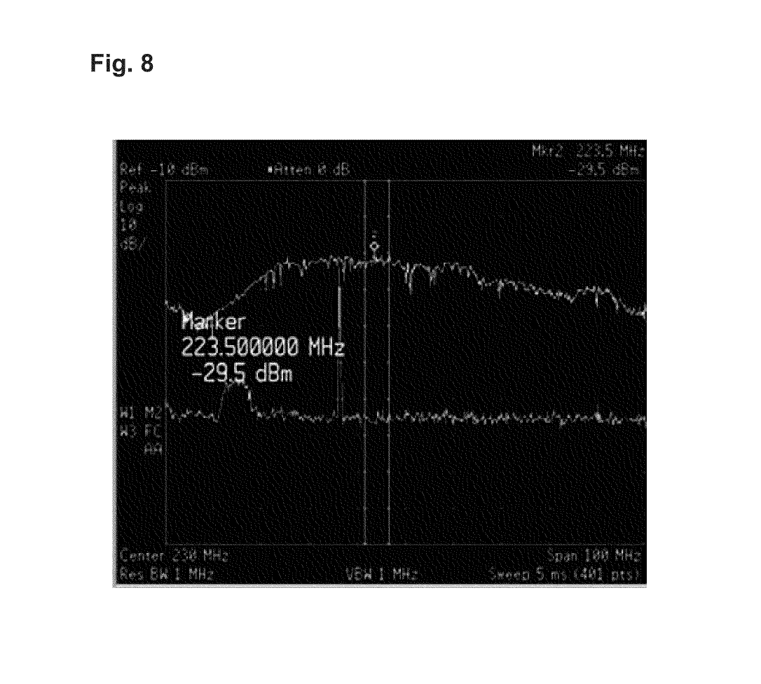

Further investigations determined that the majority of emissions were identified at 223.5 MHz, as shown in FIG. 8. It also was determined that most of the interference was emitted from the amplifier printed circuit boards (PCB) at the area where mosfet transistors are located. This was determined using a close field probe. A majority of the interference was generated due to the high speed switching of high voltage on mosfet transistors. Overshoot and undershoot spikes on the pulse as well as high frequency oscillations right after the spikes produced broadband emissions during switching periods from high to low and low to high voltage signals.

In regards to electromagnetic compatibility (EMC), there are two basic approaches to reduce the radiated emissions: First, to find the source of the emissions and design the circuits in the fashion that will reduce the emissions as much as possible or eliminate it completely. Second, to shield the internal sources of the radiated emissions so that emissions are not present outside of the device. The first approach can be a better approach if emissions can be completely eliminated, but in most cases emissions could only be reduced and therefore both approaches have to be utilized. A signal switching amplifier, as described above, utilizes both approaches.

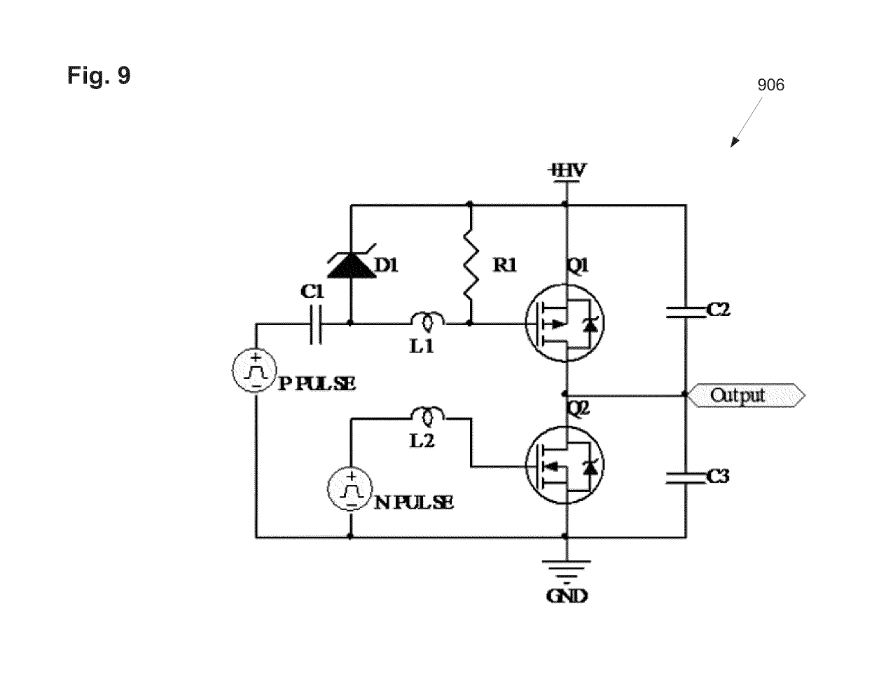

It was also determined that the edges of the pulses need to be “dull” and free of high frequency oscillations from spikes during high speed, high voltage switching on the mosfet transistors. Two important changes to the channel amplifier PCB at the area of the mosfet transistors were made. Referring to FIG. 9, which illustrates amplifier 906 (corresponding to amplifier 206 from FIG. 2):

First, capacitors C2 and C3 were added at the output of the mosfet transistors Q1 and Q2 but before the matching network. In one embodiment, these capacitors can be 330 pF 1000V ceramic SMD1206 capacitors. The value of the capacitors should be very small (in pF, pico farads) so that output of the amplifier is not “loaded”. Exact value can depend of operating frequency and level of noise that needs to be suppressed.

Second, inductors L1 and L2 were added on the gates of all the mosfet transistors. In one embodiment, these inductors can be ferrite inductors SMD1806 470 ohm@100 MHz. It is preferable to position the inductors as close to the gates as possible. The emission suppression value of the inductor has to be chosen carefully in order to have a good balance between emission suppression and efficiency of the amplifier. Too low value may not be enough to suppress the emissions, and too high value can expend switching time of the mosfet causing overheating and inefficiency. In some embodiments, values higher than 470Ω at 100 MHz can make switching time longer, which can cause extra heat dissipation and inefficiency. Lower values can reduce noise. The addition of the capacitors C2 and C3 and the inductors L1 and L2 to the amplifiers of the Histotripsy therapy system can be referred to herein as a radiation emission suppression circuit.

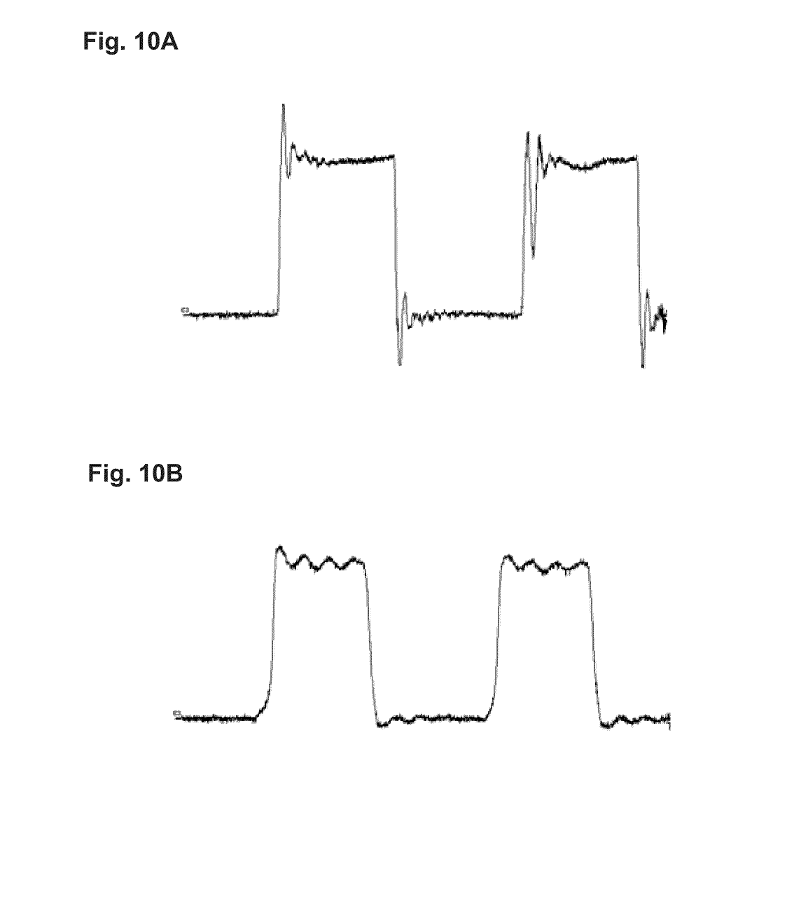

These changes to the mosfet amplifier circuit eliminated the spikes at the beginning and end of the square wave pulse output. FIG. 10A shows the square wave pulse output before adding the capacitors and inductors of the circuit in FIG. 9. FIG. 10B shows the square wave pulse output after adding the capacitors and inductors of the circuit in FIG. 9. The spike at the beginning and end of the pulse output is clearly minimized or eliminated completely in the plot of FIG. 10B.

Other changes were made to reduce emissions at lower frequencies, including enclosing the entire therapy generator (amplifier) into a metal housing to keep air clearance and creepage distances to satisfy isolation and safety requirements, and tuning the main operating frequency to the anti-resonant frequency of the transducer. It was found that shielding is more effective on higher frequencies (above 200 MHz) than on lower.

With both approaches combined, radiated emissions were reduced to the acceptable levels.

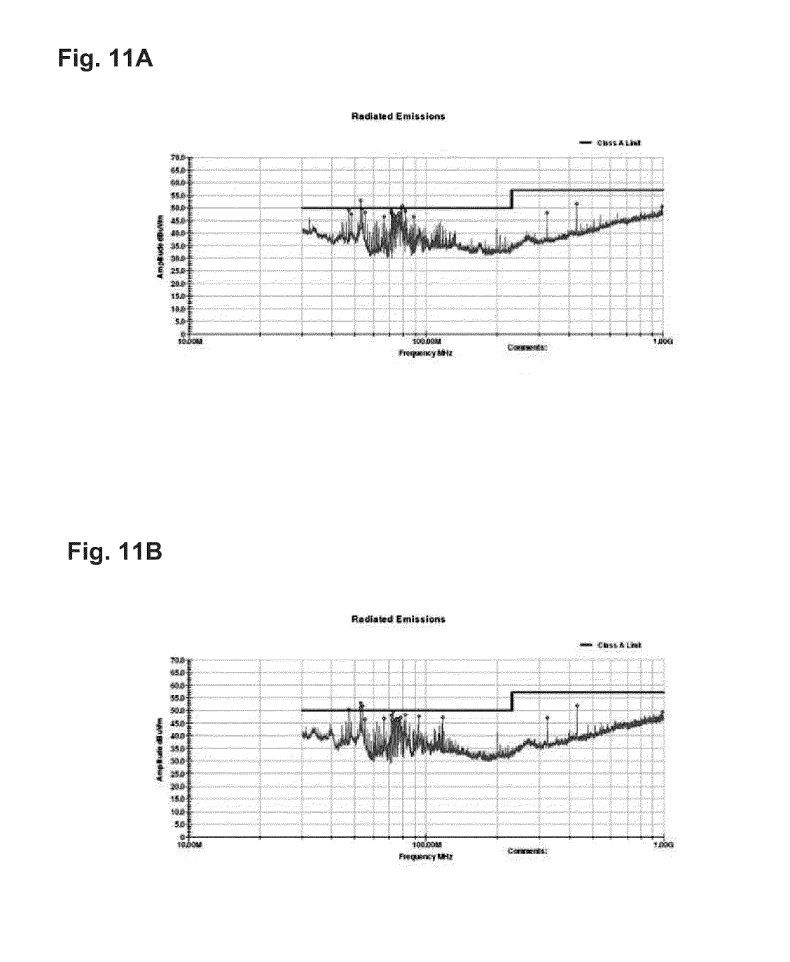

The system was retested at a certified testing lab after circuit board redesign and other changes were made. The revised system met all IEC60601-1 standards including EMC compliance. This is shown in FIGS. 11A-11B. Compare the results to FIGS. 7A-7B, and it can be seen that with the radiation emissions in the normal mode of operation no longer exceed the Class A limit.

Histotripsy transducer dimensions, including aperture (diameter) and focal length, can be established based on the clinical application requirements. With Histotripsy therapy, the optimal f-number (aperture/focal length) is between 0.8-1. Transducers with f-numbers close to one are able to concentrate the acoustic energy efficiently to reduce the effects of attenuation and non-linearity caused by the tissues. F-numbers lower than 0.8 may affect the ability to move the focus through axial plane (i.e., the working distance). The lower f-numbers can be effective if axial movement is not required. Larger f-numbers can cause heating in tissues between the skin surface and target and must be avoided.

In general, larger aperture transducers are necessary to treat tissues that are deep in order to accommodate a longer focal length. Small transducers can be used to treat more superficial tissues non-invasively, endoscopically or intra-operatively as they have shorter focal lengths with small apertures.

Transducers can be designed with one or more elements, as described above; however, according to some embodiments, the impedance of each element should be more than 100Ω. The high impedance of the transducer elements matched with low output impedance of the amplifier is essential to create substantial voltage gain in the matching network.

According to the present invention, a single amplifier and with matching network can be applied to each element of the Histotripsy therapy transducer. Driving multiple transducer elements with the same amplifier reduces the transducer impedance and subsequently reduces the voltage gain achieved in the matching network oscillation circuit. More than one transducer element can be driven by the same amplifier as long as the combined impedance is more than 100Ω. The output impedance of the amplifier can be between 5 and 30 ohms. In some embodiments, the input impedance of the transducer should be at least 2 times higher than the output impedance of the amplifier.

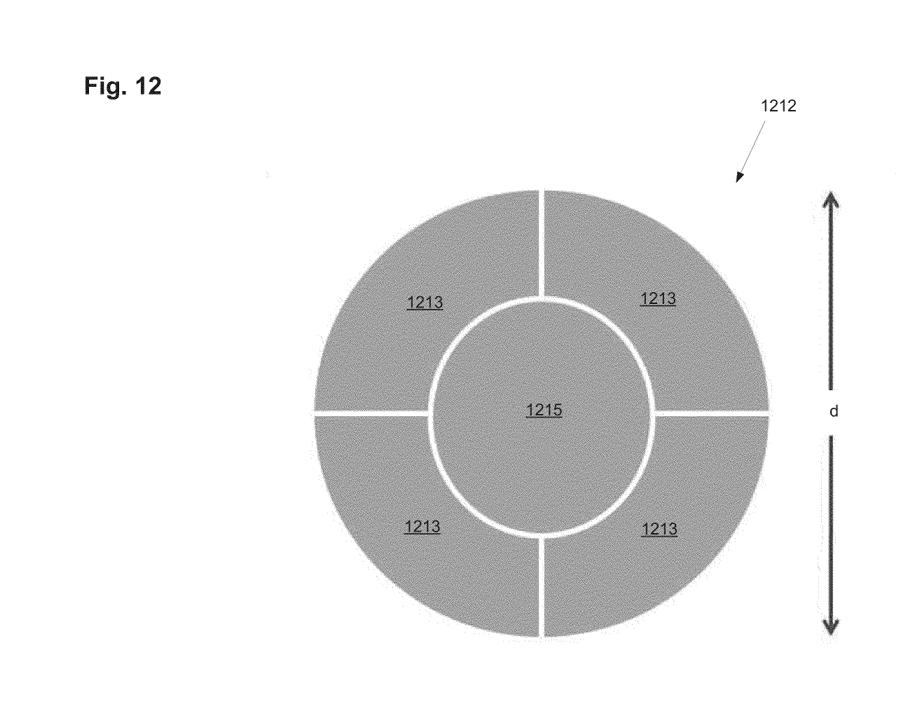

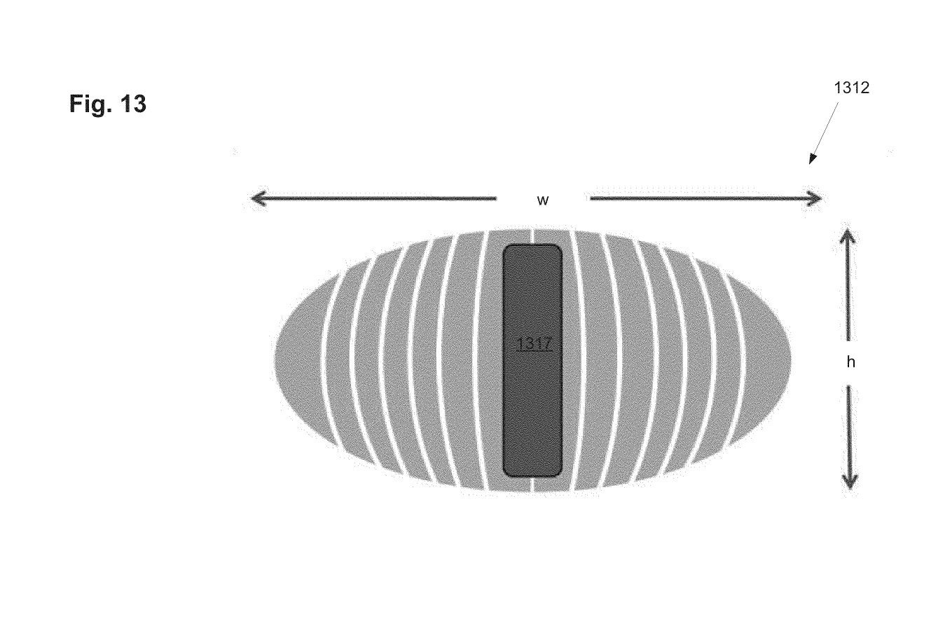

Superficial lesions such as tumors of the thyroid gland can be treated non-invasively with Histotripsy therapy systems that utilize a transducer with a shorter focal length. FIG. 12 shows one embodiment a transducer 1212 configured to treat thyroid tumors. In some embodiments, this transducer can be used to treat thyroid tumors at depths from 1-4 cm and can have a 5 cm diameter d and 5 cm focal depth. The transducer can comprise multiple elements 1213 arranged racially around the circumference and a central round element 1215. In one specific embodiment, each element can be 3.7 cm² with an impedance of approximately 100Ω. Each of the 5 transducer elements can be driven with a separate amplifier and matching network as described in the above embodiments.

FIG. 13 illustrates yet another embodiment of a Histotripsy transducer 1312 configured to perform Histotripsy therapy. The Histotripsy can be used endoscopically, laparoscopically, or intraoperatively and delivered with elliptical phased array transducer 1312. In some embodiments, the middle of the transducer has an opening 1317 comprising a linear ultrasound imaging transducer. The phased array enables the transducer to vary the focal length to change the treatment depth and position. As shown, the elliptical transducer comprises a plurality of transducer elements. In some embodiments, the transducer can include 14 transducer elements each having an area of approximately 1 cm². The impedance of these transducer elements can be 370Ω (note that 3.7 cm² elements had impedance of 100Ω). The elliptical shape of the transducer can be defined by a height h and width w.

As in the embodiments described above, each element can be driven by a separate amplifier. In some embodiments, a matching network circuit can match the 370Ω of the transducer to the 20Ω output of an amplifier. Alternatively, a single amplifier can drive two transducer elements preferably at opposite ends of the transducer for variation of focus depth in the phased array. In this embodiment, two elements driven in parallel would an input impedance of 185Ω.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A histotripsy therapy system, comprising:
at least one signal switching amplifier electrically coupled to a high voltage power supply;
a pulse generator electrically coupled to the at least one signal switching amplifier;
at least one matching network electrically coupled to the at least one signal switching amplifier; and
an ultrasound transducer having at least one transducer element, each transducer element of the ultrasound transducer being coupled to the at least one matching network, each transducer element having an input impedance that is more than 2 times higher than an output impedance of its corresponding signal switching amplifier.

2. The system of claim 1 wherein the system is configured to provide histotripsy therapy comprising forming cavitational microbubbles within tissue at a focal zone of the transducer without damaging surface tissue.

3. The system of claim 1 wherein the output impedance of the signal switching amplifiers is less than or equal to 50 ohms.

4. The system of claim 1 wherein the input impedance of each transducer element of the ultrasound transducer is between 10 and 500 ohms.

5. The system of claim 1 wherein the pulse generator provides a square wave voltage of up to 1000V.

6. The system of claim 5 wherein the plurality of signal switching amplifiers convert the square wave voltage into a sinusoidal wave voltage that is 2 to 10 times greater than the square wave voltage.

7. The system in claim 6 wherein the sinusoidal wave voltage is up to 5000 Vpp.

8. The system of claim 1 wherein the at least one transducer element is configured to be driven at up to 4 MHz.

9. The system of claim 1 wherein the at least one matching network is configured to match or exceed the output impedance of the at least one signal switching amplifier with a combined impedance of the at least one matching network and at least one transducer element.

10. The system of claim 1 wherein the ultrasound transducer has a diameter of ranging from 2 cm to 50 cm.

11. The system of claim 3 further comprising an emission suppression circuit that includes inductors placed in series with gates of mosfet transistors disposed in the at least one signal switching amplifier, and further includes capacitors placed in parallel with an output of the at least one signal switching amplifier, the inductors and capacitors being configured to reduce electromagnetic emissions of the at least one signal switching amplifier.

12. The system of claim 11 wherein the inductors and capacitors are configured to eliminate spikes at a beginning and end of a square wave pulse.

13. The system of claim 1 wherein the ultrasound transducer has an F-number between 0.4 and 1.2.

14. A Histotripsy therapy system, comprising:
at least one signal switching amplifier electrically coupled to a high voltage power supply;
a pulse generator electrically coupled to the at least one signal switching amplifier;
at least one matching network electrically coupled to the at least one signal switching amplifier; and
an ultrasound transducer having at least one transducer element, each transducer element of the ultrasound transducer being coupled to the at least one matching network, each transducer element having an input impedance that is higher than an output impedance of its corresponding signal switching amplifier so as to deliver a Histotripsy pulse having a pulse length less than 20 μsec, a peak negative pressure greater than 10 MPa, and a duty cycle less than 5% to tissue.

15. The system of claim 14 wherein the output impedance of the at least one switching amplifier is less than 50 ohms.

16. The system of claim 14 wherein the input impedance of the at least one transducer element is greater than 50 ohms.

17. The system of claim 14 wherein the at least one matching network matches a real impedance of the at least one transducer element to the output impedance of the at least one signal switching amplifier and reduces an imaginary impedance of the at least one transducer element.

18. An ultrasound therapy system, comprising:
a high voltage power supply;
a pulse generator;
a plurality of signal switching amplifiers electrically coupled to the high voltage power supply and the pulse generator;
a plurality of matching networks, each matching network being electrically coupled to one of the plurality of signal switching amplifiers; and
an ultrasound transducer having a plurality of transducer elements, each transducer element of the ultrasound transducer being electrically coupled to one of the plurality of matching networks and one of the plurality of signal switching amplifiers, wherein each transducer element has an input impedance that is higher than an output impedance of its corresponding signal switching amplifier.

19. The system of claim 18 wherein the input impedance of each transducer element is at least 2 times higher than the output impedance of each corresponding signal switching amplifier.

20. The system of claim 18 wherein the input impedance of each transducer element is at least 100Ω and the output impedance of each signal switching amplifier is less than or equal to 50Ω.

21. The system of claim 18 wherein the ultrasound transducer comprises an F-number less than 1.2.

22. The system of claim 18 further comprising 36 signal switching amplifiers, 36 matching networks, and 36 transducer elements.

23. The system of claim 18 wherein the plurality of matching networks are configured to bring the combined impedance of the plurality of matching networks and the plurality of transducer elements down to match that of the plurality of signal switching amplifiers.

24. The system of claim 18, wherein the plurality of signal switching amplifiers each include an emission suppression circuit configured to reduce spikes on a square wave pulse output of the signal switching amplifiers.

25. A method of delivering Histotripsy ultrasound therapy, comprising:
generating in a pulse generator a Histotripsy pulse having a pulse length less than 20 μsec, a peak negative pressure greater than 10 MPa, and a duty cycle less than 5%;
amplifying the Histotripsy pulse from the pulse generator with a plurality of signal switching amplifiers;
impedance matching an input impedance of a plurality of ultrasound transducers to an output impedance of the plurality of signal switching amplifiers with a plurality of matching networks; and
delivering the Histotripsy pulse to tissue with the plurality of ultrasound transducers.
